(19) United States
(12) Patent Application Publication
(10) Pub. No.: US 2001/0044590 A1
(43) Pub. Date: Nov. 22, 2001

(54) Orthopedic braces having sublimated graphics

(76) Inventors: Dylann D. Ceriani, San Diego, CA (US); Del R. Doty, Cardiff by the Sea, CA (US); Scott P. Seligman, Carlsbad, CA (US); Richard E. Gildersleeve, Escondido, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/815,774

(22) Filed: Mar. 22, 2001

Related U.S. Application Data
(62) Division of application No. 09/326,384, filed on Jun. 4, 1999, now abandoned.

(51) Int. Cl. A61F 5/00

(52) U.S. Cl. 602/26; 602/23

(57) ABSTRACT

A method for sublimating graphics onto a three-dimensional object, such as an orthopedic brace, includes pre-treating the device if needed. The device may require a coating that is able to receive sublimated inks and capture those inks. Sublimation papers are cut to the appropriate size, placed on the device with re-positional adhesive, and wrapped around the object. Shrink tubing and clamps may also be used to ensure continuous contact between the sublimation paper and the device during sublimation. After the sublimation paper has been placed against the desired surfaces of the device, the paper and the device are heated to sublimate the inks into the device. After the device and paper are heated and allowed to cool, the sublimation paper is removed with a lubricant or solvent. Orthopedic devices and other three-dimensional objects having a complexity in their geometric profile can therefore be provided with sophisticated graphics. The user of the orthopedic device can select the graphics to appear on the orthopedic device and even may provide the graphics in the form of a drawing, photograph, or file.

### FIELD OF THE INVENTION

The present invention relates generally methods for sublimating graphics and articles having sublimated graphics and, more particularly, to methods for sublimating graphics on orthopedic devices.

### BACKGROUND OF THE INVENTION

Sublimation graphics result in high-resolution, near photographic images that constitute an alternative to screen printing or other graphics imparting processes on various materials. The term sublimation, which is used in chemistry, refers to the phenomenon of a substance passing from its solid state to a gaseous state and vice versa without passing through a liquid state, as is the case for “dry ice.” Thus, sublimation graphics is a process that involves a type of dye which, when heated to a certain temperature, converts to a gaseous state from its former solid state. The dye molecules in the gaseous state then penetrate the surface of a receiving substrate to be printed. After the heat source is removed, the sublimated dyes permanently bond to the substrate and the result is a continuous tone, high-resolution image embedded in the surface of the receiving item.

Sublimation is used extensively to produce images on textiles where detailed colors and fade-resistant graphics are desired. Polyester fabrics are very well suited for receiving sublimation, while cotton fabrics do not yield good results. Images produced by sublimation processes on coated ceramics, metals, and glass are bright, of high gloss and color, and cannot easily be scraped off because they are incorporated in the coating of the receiving item.

The sublimation dyes are transferred to the receiving item via a paper carrier. During sublimation, the dye transfer paper is applied to the receiving substrate and the combination is heated with a heat mold, press, or other methods to a transition temperature at which the substrate can receive the sublimation gas molecules. This temperature is known as the glass transition temperature for polymers and at or above this temperature polymers become soft but do not melt. When polymers reach the glass transition temperature, the molecular chains or strands relax or expand, whereby they skew to form openings that receive the individual gaseous dye molecules. When the material is cooled after completion of the sublimation process, the polymers regain their original shapes and envelop the dye molecules which have solidified inside the polymers. If the glass transition temperature of the substrate being printed is not reached or maintained for an appropriate length of time, much or all of the sublimation dye will remain on the surface of the substrate and the image becomes subject to fading, bleeding, and smearing. Additionally, if the dyed substrate later is brought to a temperature above its glass transition temperature, for example to apply a heat transfer decal, the polymer molecular chains relax or expand and some of the sublimated dye molecules escape, thereby considerably subduing the high resolution and bright colors of the transfer image.

There are two general types of sublimation techniques, which primarily differ by the type of dye transfer paper used. These techniques include thermal transfer sublimation and true sublimation. The thermal transfer sublimation category includes dye sublimation and wax thermal sublimation. Dye sublimation refers to a process whereby printers sublimate primary colors, such as cyan, magenta, yellow, and black, onto the paper carrier. The color tones are controlled by varying the duration of the heating process for each of the primary colors that are blended together for each pixel of the printed image. When the transfer paper subsequently is heat pressed onto the item to be printed, the dyes are resublimated onto the receiving substrate. Wax thermal sublimation refers to a process whereby dye pigment is suspended in a wax medium on printing ribbons. The dyes are applied to the paper carrier by melting the wax carrying the appropriate dyes at a temperature high enough to melt the wax, but not so high that the dye sublimates. Typically, various colors are generated through a half-toning or dithering process, whereby one of three primary dye colors, such as cyan, magenta, or yellow, is applied to individual pixels so that when viewed together with neighboring pixels, form the illusion of different hues depending on the color combinations of neighboring pixels.

For the true sublimation process, the sublimation transfer paper usually is produced by saturating uncoated calandered paper with sublimation dyes in offset or lithographic presses. Sublimation transfer paper graphics can also be acquired from dye-sublimation color printers. Thus, heat is only applied in the actual process of sublimating the dyes from the transfer paper to the receiving substrate. As a result, true sublimation graphics have very high resolution finishes and are very well suited for high-volume sublimation tasks.

Not every surface can be sublimated. The typical sublimation dye must be heated to a rather high temperature, such as approximately 248° F. This temperature is the earliest point at which the dyes that are currently used for sublimation begin to convert to a gaseous state. At this temperature, however, the sublimation process can be rather slow. As the temperature is increased above 300° F., the conversion to the gaseous state occurs more rapidly, and at 400° F. it is virtually instantaneous. Although the sublimation of the dye itself takes place very rapidly, it takes anywhere from 20 seconds to a number of minutes to complete the transfer process because additional time may be required to bring the receiving substrate to a temperature at which the substrate can receive the ink. These high temperatures for sublimating the graphics and also the length of time that the substrate must be exposed to these temperatures are prohibitive for many surfaces.

While conventional sublimation graphics methods function well for two-dimensional receiving substrates, such methods are not readily applied to three-dimensional objects particularly those having complex surface geometries, such as orthopedic devices. One reason is that conventional heat presses designed for two-dimensional objects cannot be used to bring the dye transfer paper and receiving synthetic substrate to their respective sublimation and glass transition temperatures on three dimensional objects. Heat molds have been designed for sublimating graphics onto mass-produced uniform-shaped three-dimensional items, such as coffee mugs, but cannot practically be used with many objects that have varying shapes or sizes due to the large number of molds that would be required. For instance, some orthopedic devices are custom made and are tailored to fit the user, whereby three-dimensional heat molds of uniform sizes and/or shapes cannot be used. In fact, it has been estimated the Defiance ACL (anterior cruciate ligament) brace of DonJoy of Vista, Calif., can be customized to have any one of over 18 billion different shapes.

Additionally, sublimating graphics onto three-dimensional objects with complex surface geometries while achieving continuous, high quality images particularly around the complexities within such surfaces is difficult. The sublimation process depends on maintaining direct contact between the dye transfer paper and the receiving substrate. While dye transfer paper is easily applied to two-dimensional or mass produced three-dimensional substrates through the use of heat presses or heat molds, the dye transfer paper is not easily maintained in contact with surfaces of three-dimensional objects with complex surface geometries.

Furthermore, another difficulty in sublimating graphics onto three-dimensional objects is that the shape of the objects may be deformed during the heating process. In conventional sublimation graphics procedures, a temperature higher than that of the glass transition point for the receiving synthetic substrate or coating or the sublimation point for the dye simply results in a shorter dye transfer time. While this remains true for three-dimensional objects, a higher temperature also increases the risk that the object will become deformed, especially if the object has a glass transition temperature near the sublimation temperature. This difficulty is especially problematic with orthopedic devices since orthopedic devices must maintain their shape in order to properly fit the end user and properly perform their intended functions.

Because of these difficulties in sublimating graphics onto three-dimensional objects, many three-dimensional objects are limited in the types of graphics that may be applied to them. Many non-uniformly sized objects simply do not incorporate any sublimated graphics. Those objects that do have sublimated graphics are typically uniformly sized or have parts that easily accommodate sublimated graphics. Some objects, for instance, may have parts that are standard sized objects or which present a substantially two-dimensional surface. The graphics are applied to these parts and then these parts are coupled to the rest of the object. Three-dimensional objects therefore typically do not have any sublimated graphics or have sublimated graphics in only discrete surfaces of the object.

While the orthopedic device is primarily functional, the aesthetic aspect of the device cannot be overlooked. Orthopedic devices are typically worn for a long period of time, often weeks if not months. During the time that the device must be worn, the user of the device may be more willing to wear an aesthetically pleasing device, thus receiving the intended benefit of the device.

Because of the difficulties in sublimating graphics onto three-dimensional objects, efforts in improving the aesthetic aspect of orthopedic devices have generally been limited to decals, laminates, custom air-brushing, and new paints or coatings. These aesthetics offer new looks but do not satisfy the demand for more sophisticated graphics. While such aesthetic improvements are sought for all types of orthopedic devices, the need for more sophisticated graphics imaging is particularly strong for pediatric and sports applications. For example, some of the stigma and anxiety that children associate with the use of orthopedic devices may be alleviated with contemporary graphic motifs that cannot readily be applied with conventional techniques. Likewise, athletes may want flashy brilliant graphics on braces they wear.

### SUMMARY OF THE INVENTION

The present invention addresses the problems described above by providing methods for sublimating graphics onto three-dimensional objects. According to a preferred embodiment, the object for receiving the sublimated graphics is provided with a surface suitable for receiving the sublimated inks. The object may already have such a surface but, if it does not, the object is provided with a synthetic coating, such as a polyurethane. Sublimation paper carrying the sublimation inks is then applied to surfaces of the object and secured in place. A preferred way of placing the sublimation paper onto the object is with a re-positional adhesive and is further secured with shrink tubing and clamps. The object is then heated to a temperature sufficient to sublimate the inks into the surfaces of the object. After the inks have been sublimated, the sublimation paper is removed from the object along with any shrink tubing or clamps. To remove re-positional adhesive and any remaining paper from the object, the object is preferably cleaned with a lubricant or solvent.

The preferred object for receiving the sublimated graphics is an orthopedic device. An orthopedic device encompasses many different types of devices, including but not limited to braces and supports. These devices may be used prophylactically to prevent injury, as a support or means for providing pain reduction in a diseased or otherwise deficient joint, as a conservative treatment in lieu of surgery, or may be used after a medical procedure, such as surgery wherein the device is used to protect the reconstruction of an anterior cruciate ligament (ACL). The devices moreover may limit movement about a joint or, on the other hand, may completely prevent any movement. With the invention, orthopedic devices are able to incorporate sophisticated graphics that previously were not practically possible. For example, a knee-brace may incorporate graphics on a cuff to be secured to a thigh of the user and to a cuff to be secured to the calf of the user. The graphics can be applied to outer visible surfaces of the cuff as well as to interior surfaces that are hidden from view when the brace is worn.

The invention is not limited to any particular sublimation technique, such as thermal transfer and true sublimation, nor is it limited in the type of graphics that may be applied to the object. The invention, for example, may incorporate graphics supplied by the manufacturer or may include graphics which are provided from a third party, such as a user of the orthopedic device. The user consequently may provide desired graphics in the form of artwork, photographs, files, or in some other manner and these graphics can then be incorporated into the orthopedic device.

Accordingly, it is an object of the present invention to provide three-dimensional objects that incorporate sublimated graphics.

Another object of the present invention is to provide orthopedic devices, such as knee braces, that incorporate sublimated graphics.

A further object of the present invention is to provide methods for sublimating graphics onto three-dimensional objects.

Yet another object of the present invention is to provide methods for sublimating graphics onto objects while minimizing deformation or distortion of the objects.

A further object of the present invention is to provide methods for sublimating graphics onto objects having varying shapes and sizes.

A still further object of the present invention is to provide a greater variety of options to the users in the aesthetic appearance of orthopedic devices.

Other objects, features, and advantages of the present invention will become apparent with respect to the remainder of this document.

### BRIEF DESCRIPTION OF THE DRAWINGS

The accompanying drawings, which are incorporated in and form a part of the specification, illustrate preferred embodiments of the present invention and, together with the description, disclose the principles of the invention. In the drawings:

FIG. 1 is a perspective view of an ACL brace without sublimated graphics;

FIG. 2 is a perspective view of an ACL brace having sublimated graphics;

FIG. 3 is a flow chart of a method for sublimating graphics onto a three-dimensional object, such as an upper segment of a brace; and

FIGS. 4(A) to 4(E) depict the upper segment of the brace which is being subjected to the method of FIG. 3.

### DETAILED DESCRIPTION

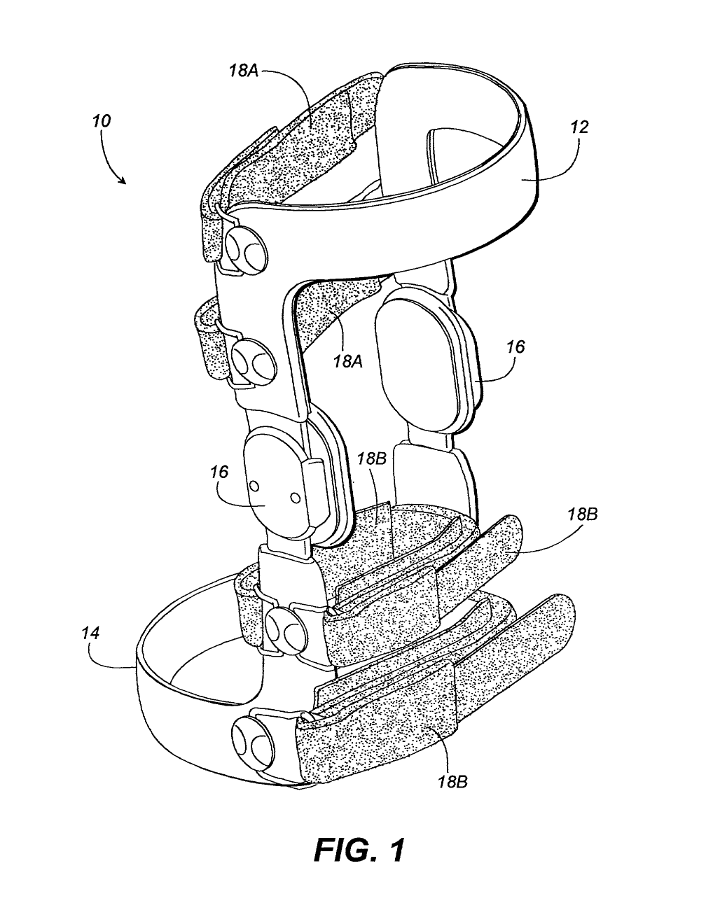

Reference will now be made in detail to preferred embodiments of the invention, non-limiting examples of which are illustrated in the accompanying drawings. With reference to FIG. 1, preferred methods according to the invention will be described with reference to an orthopedic device 10. In this example, the orthopedic device 10 is a Defiance ACL brace manufactured by DonJoy of Vista, Calif. The brace 10 illustrates the difficulties in sublimating graphics onto the brace 10 since, as discussed in the Background section of the application, the brace 10 may be customized to have any one of more than 18 billion different shapes. The invention, however, is not limited to just this type of orthopedic device but may also be applied to other orthopedic devices as well as other three-dimensional objects.

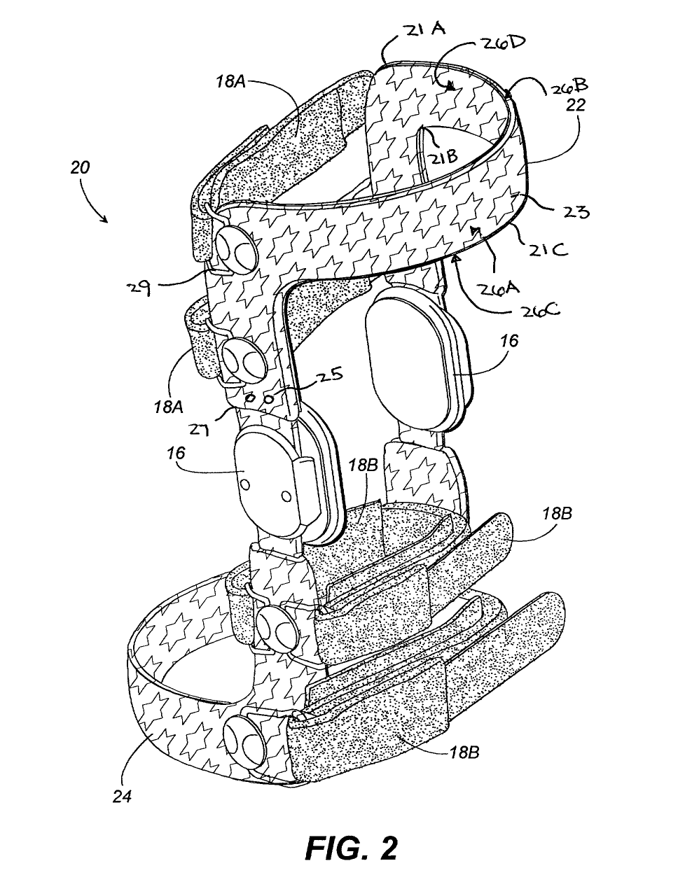

The methods and techniques according to the invention, which will be described in detail below, are well-suited to sublimate graphics into any object having a geometric profile with at least one complexity. These complexities in geometric profile include, but are not limited to, complicated, sophisticated, asymmetrical, and irregular shapes that render it difficult or impractical to use conventional heat presses or molds to sublimate the graphics. The complexity, for instance, may be a concave surface on an object or may be the result of the desire to place sublimated graphics on a combination of surfaces which may include outer, side, and/or inner surfaces of an object or to wrap around from the inner to the outer surface of the object. The complexity may also be the result of objects having non-uniform shapes and/or sizes. As discussed above, some orthopedic devices are customized to fit a particular user whereby the devices exhibit variation in shapes as well as in sizes. This variance renders it impracticable to sublimate graphics onto the orthopedic devices with conventional heat presses or heat molds. More specific examples of such complexities are illustrated in FIG. 2, wherein the brace 20 is shown having a surface 23 that has a number of complexities, including rivet 25, interface 27, D-ring 29, and curves 21A, 21B, and 21C. Other geometric profiles having at least one complexity will be apparent to those skilled in the art and are encompassed by the invention.

The brace 10 is generally comprised of two cuffs, generally referred to as an upper segment 12 and a lower segment 14, and also joints 16 for coupling the two segments 12 and 14 together. The upper segment 12 is intended to be secured to the upper leg of the user through straps 18A and the lower segment 14 is intended to be secured to the lower leg of the user through straps 18B. The joints 16 preferably control movement of the user's legs, for instance by limiting flexion or extension of the leg or by preventing strain on ligaments of the knee.

The knee brace 10 has been generally limited in the type of graphics that may be applied to it. The upper and lower segments 12 and 14 may be painted in a variety of colors and decals may be secured to the outer surfaces of these segments 12 and 14. Sublimated graphics, on the other hand, have not been applied to this brace 10 due to the difficulties discussed in the Background section as well as other obstacles encountered in sublimating graphics onto three-dimensional objects.

An example of a three-dimensional object 20 having sublimated graphics is shown in FIG. 2. The object 20 is a Defiance ACL brace from DonJoy and, unlike the brace 10 shown in FIG. 1, incorporates sublimated graphics. The brace 20 includes an upper segment 22 that is intended to be secured to the upper leg of a user with straps 18A and a lower segment 24 that is intended to be secured to the lower leg of the user with straps 18B. As shown in FIG. 2, both the upper segment 22 and the lower segment 24 have graphics that are applied on inner, outer, and side surfaces of the segments 22 and 24. As will be described in more detail below, the graphics have been sublimated onto the segments 22 and 24.

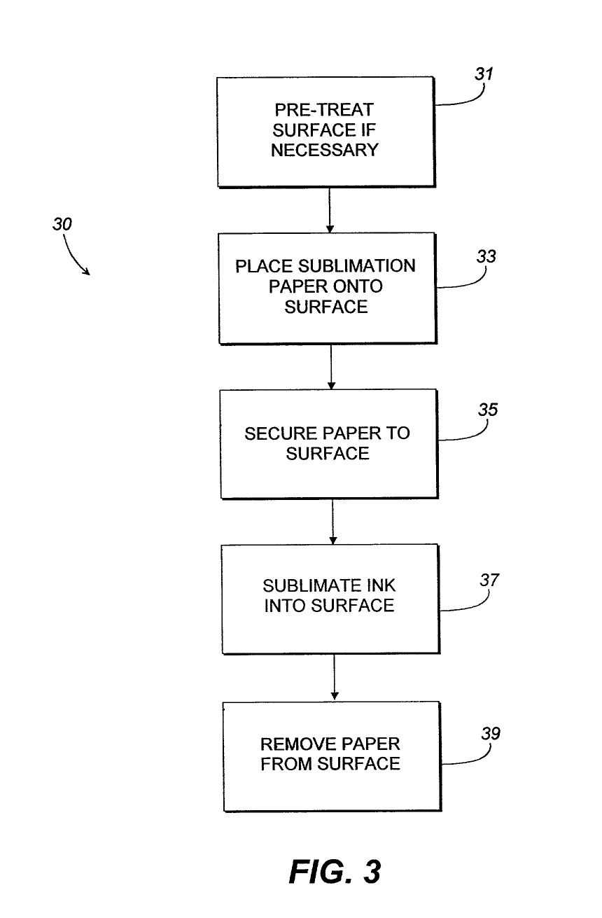

FIG. 3 generally illustrates a method 30 for sublimating graphics onto a three-dimensional object, such as the brace 20. At 31, surfaces of the three-dimensional object to receive the sublimated images are pre-treated if necessary. To receive sublimated graphics, the surface must be comprised of molecules that are able to relax or expand so that sublimated ink may be received and retained. Consequently, for some objects, a coating may need to be applied at 31. In accordance with the preferred embodiment of the present invention, a synthetic coating is used that is preferably a polymer, and most preferably a polyester-based polymer.

At 33, sublimation paper is placed onto the surface 23 of the object. The sublimation paper is preferably placed on the surface 23 so that substantially continuous intimate contact occurs between the sublimation paper and all complexities within the surface 23. This intimate contact can be accomplished through the use of adhesive on the paper and/or pressure applied to the paper. At 35, the paper is secured to the surface and then at 37 the ink is sublimated into the surface. At 37, the ink and the surface are heated to a temperature at which the ink sublimates and moves from the paper carrier into the surface. The temperature must also be high enough to cause the molecules in the structure to be able to receive the sublimated ink. At 39, after the object is cooled, the paper carrier is removed from the surface and any device, mechanism, or material used at 35 to secure the paper to the surface is also removed. Through this method 30, sublimated graphics can be applied to the object with virtually no distortion in the desired design on visible surfaces despite complexities in the geometric profile of the object. Thus, virtually distortionless graphics can be applied to objects having complicated, sophisticated, asymmetrical, concave, or irregular shapes.

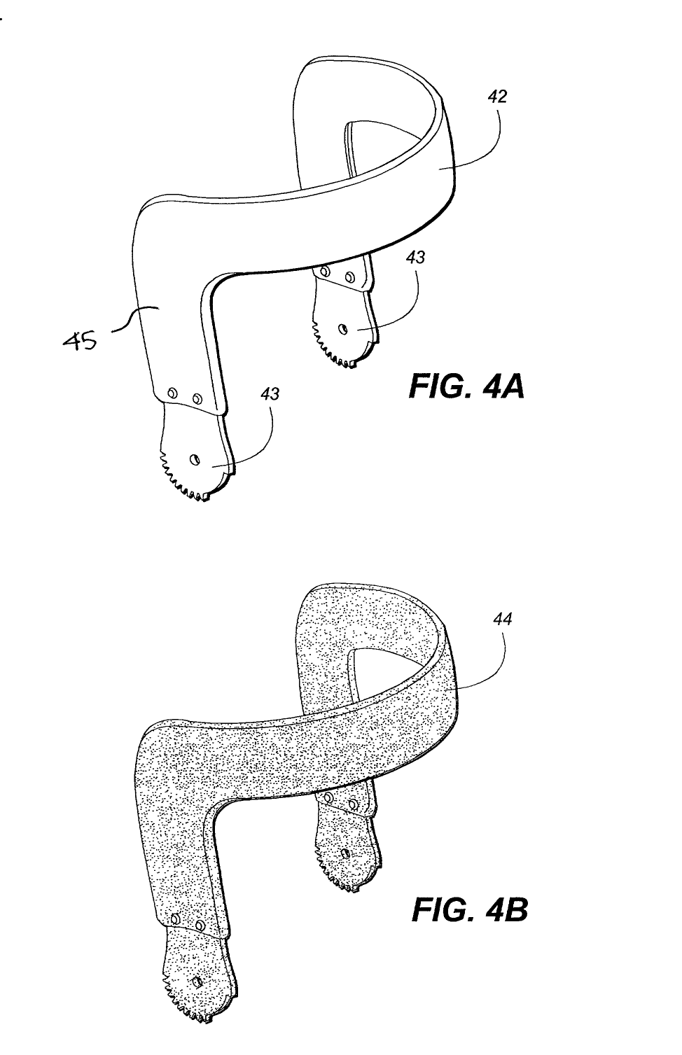

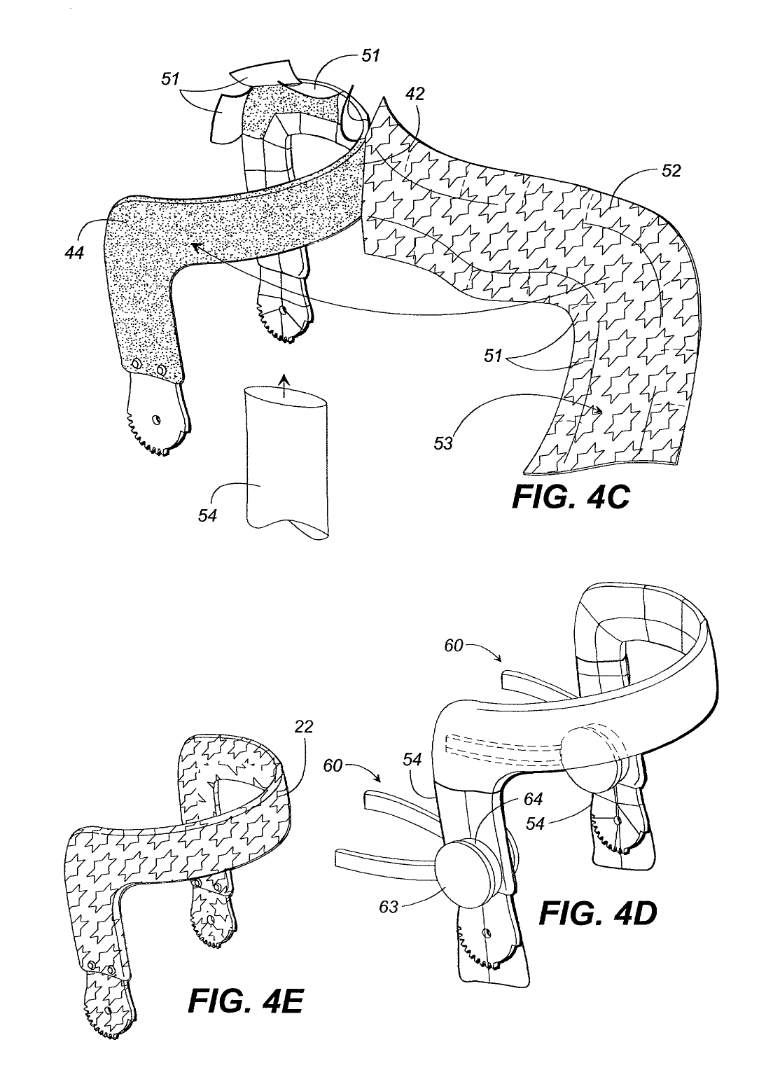

An example of the preferred method 30 will now be described with reference to FIGS. 4A to 4E. FIG. 4A illustrates an un-treated upper segment 42 of the brace 20. The upper segment 42 includes a leg support portion 45 and a geared hinge portion 43. The geared hinge portion 43 is designed to form part of the joints 16, and is preferably made from a metal such as Aluminum 6061-T6. The leg support portion 45 is formed of carbon fiber epoxy composite joined to the hinge portion 43. Because the carbon fiber epoxy composite and the metal do not readily accept sublimated inks, they cannot be used as the sublimating receiving surface. Therefore, the upper segment 42 is pre-treated by applying a coating 44, as shown in FIG. 4B. The coating 44 will serve as the sublimation receiving surface, and may comprise any suitable coating that is able to receive sublimated inks, such as polyurethane paint, powder-coating, or other synthetic coatings. In the examples described herein, polyurethane paint is used as the coating 44. Those skilled in the art will readily appreciate that in certain embodiments of the present invention the sublimation receiving surface will consist of the outer surface of the material from which the upper segment 42 is fabricated. This is true for embodiments wherein the upper segment 42 is formed from materials such as polyurethane, acrylonitrile, butadiene, and styrene.

In pre-treating the sublimation receiving surface at 31, one factor that should be considered when sublimating graphics onto three-dimensional objects is moisture. During the sublimation process, moisture converts into a vapor state much sooner than the sublimation dyes convert to their gaseous state. As a result, the water molecules fill openings in the receiving surface thereby blocking the transfer of the dye into the surface. If the sublimation dyes are unable to penetrate the receiving substrate, the dyes are forced to rest on the outside surface, making the image less permanent and subject to smearing, bleeding, and fading. To facilitate the removal of moisture in or on the receiving surface, the object is preferably pre-heated to a temperature above the boiling point of water, approximately 212° F., for a sufficient amount of time, approximately 10 to 20 minutes.

The sublimation receiving surface should be durable in addition to providing a surface conducive to sublimation. Moreover, the sublimation receiving surface can be tailored to produce sublimated graphics of differing intensities and gloss. For example, colored synthetic coatings, such as polyurethane paints and polyester powder coats, can be used as coating 44 to produce muted, low-key graphics. Alternatively, clear coating can be applied over a colored synthetic coating to produce brilliant, eye-catching images. Metal flecks or pearlescent hues may be added to the coating 44 before application to affect light refraction properties or to produce metallic appearances. Clear polyurethane paint was determined to be the preferred synthetic coating material due to its tough, scratch- and chip-resistant, high-gloss finish and ease of application in a manufacturing setting. The preferred thickness of the coating 44 was determined to be no less than two to three thousandth of an inch (mils), as coatings of lesser thickness resulted in faded or mottled transfer images. Water-borne paints were not as effective in producing brilliant images.

The pre-treatment at 31 may also involve some other acts that are performed on the object. When a clear polyurethane paint is used as the coating 44, the object may be colored prior to applying the coating 44. The color influences the appearance of subsequently sublimated graphics; light-colored backgrounds produce images with brighter colors and dark-colored backgrounds create different more subdued looks. It was determined that paint, other pre-coatings, and/or the final coating 44 applied to the devices should be pre-cured before sublimating so that the surface of the sublimation receiving surface is hard enough to resist indentations caused by clamps and shrink tubing used in securing sublimation paper to the object. The segment 42 is preferably pre-cured at 220° F. for 15 to 20 minutes and is allowed to cool completely. Pre-curing was also found to be important in allowing the sublimation transfer paper 52 to be easily removed after the inks have been sublimated into the object. The invention does not require pre-curing or additional pre-treatment. For example, it was determined that objects sublimated after the coating 44 had reached full cure, which occurred at seven days for polyurethane paint, did not need to be pre-cured.

FIG. 4C depicts sublimation paper 52 being placed on the upper segment. A preferred manner of securing the sublimation paper 52 to the upper segment 42 is with RePositional Spray Adhesive, which is manufactured by Minnesota Mining and Manufacturing Company (3M). During sublimation, the ink will pass through this adhesive with no significant effects on the overall process or the resulting product. The sublimation paper 52 is first die-cut to a general shape of the upper segment 42 with some offset portion, such as an extra inch of paper 52. The sublimation paper 52 is then applied to the upper segment 42 and is wrapped onto the surfaces of the upper segment 42. To place the paper onto the upper segment 42, adhesive may be added to the brace or to the transfer side of the sublimation paper 52. The sublimation paper 52 is preferably coated with re-positional spray adhesive so that the paper 52 may be lifted off the segment 42 and repositioned if necessary to remove any wrinkles in the paper 52. The paper 52 may already be coated with the re-positional adhesive or the adhesive may be applied to the paper 52 at the manufacturing site. The sublimation paper 52 or the upper segment 42 should not be soaked with the adhesive, but instead the adhesive should be sprayed at a distance of approximately 8 to 12 inches and allowed to dry until tacky before the paper 52 is applied to the upper segment 42.

To wrap the sublimation paper 52 onto the complexities of the upper segment 42, namely its irregular contours, portions of the paper 52 may be slit to smoothly place the paper 52 on the outer visible surfaces. Referring to FIG. 2, you will note that when the brace is positioned on a leg the surfaces that will be visible (i.e. the outer visible surfaces) consists of the front surface 26A, the upper surface 26B, and the lower surface 26C. The inner surface 26D is not visible and therefore it is not critical that the paper 52 be smoothly placed over this surface.

The slits 51 can be made during the die-cutting process or may be made with scissors at a later time. Furthermore, the paper 52 is preferably placed on the object before the slits 51 are made to allow for slight variations in sublimation paper 52 placement without disruptions in graphic continuity on the visible surfaces of the upper segment 42. The slits 51 are preferably formed along edges of the paper 52 and spaced approximately 0.5 to 1.0 inch apart to prevent wrinkling of the paper 52.

Next, as shown in both FIGS. 4C and 4D, the sublimation paper 52 is secured to the upper segment 42. Some difficulty was encountered in maintaining contact between the sublimation paper 52 and the upper segment 42 during the sublimation of the inks into the upper segment 42. As the sublimation paper 52 and the upper segment 42 are being heated, the paper 52 has a tendency to separate from the upper segment 42, thereby interfering with the application of the graphics to the upper segment 42. To prevent the sublimation paper 52 from separating from the upper segment 42, as discussed above, the sublimation paper 52 is preferably coated with the re-positional spray adhesive. Furthermore, shrink tubing 54 is placed around the upper segment 42 to maintain intimate contact between the sublimation paper 52 and the upper segment 42.

The shrink tubing 54 is preferably flat and at least 0.07 mm in thickness. The tubing 54 should be able to shrink to the minimum cross-sectional circumference of the object and, unencumbered, may possibly shrink 5-10% smaller than the cross-sectional circumference of the object to further prevent the sublimation paper from separating from the surface. In some instances, the tubing 54 may split due to high tensile stresses created after shrinking, but the splitting may be minimized by placing the seam of the tubing 54 on a relatively flat or large radius surface of the object, such as the outer, visible surface of the upper segment 42. The suggested temperature to fully shrink the preferable tubing is approximately 275° F. for an exposure time of 4-6 seconds, and the temperature may be increased if shorter cycle times are required. Likewise, the temperature may be reduced if the exposure time is increased.

Another difficulty encountered was that the sublimation paper 52 had a tendency to separate from the upper segment 42 at certain complexities within the sublimation receiving surface, including at rivets, leg support portion 45/gear portion 43 interface, and concave surfaces. To prevent lift-off at such places, clamps 60 are preferably used to force the sublimation paper 52 onto the upper segment 42. The clamps 60 may comprise any suitable clamp, preferably spring-biased, and hold a silicone foam padding 64 mounted on thin rigid surfaces 63. The clamps 60 bias the mounts 63 toward each other, thereby forcing the silicone foam 64 into firm contact with the sublimation paper 52.

Next, at 37 the upper segment 42 of the brace together with the sublimation paper 52, shrink tubing 54, and clamps 60 are heated in a convection oven or heat tunnel in order to sublimate inks into the surface of the upper segment 42. One challenge in applying sublimated graphics to an orthopedic device is the proximity of the ink sublimation temperature to that of the glass transition temperature of the orthopedic device. In this example, the inks sublimate at approximately 248° F., therefore in accordance with one embodiment the paper covered upper segment 42 is heated to a temperature of greater than 248° F. However, the glass transition temperature for the carbon fiber epoxy composite is between 260° F. and 290° F. Both the temperature at which the segment 42 and paper 52 are heated and the time that the segment 42 and paper 52 are exposed to the heat were varied in order to find a preferred temperature and time. Greater temperatures reduced the time needed to sublimate the inks into the segment 42 but increased the risk that the segment 42 may become deformed from its desired shape. On the other hand, lower temperatures required a greater time to sublimate the inks into the segment 42 but presented less risk that the segment 42 would have its shape altered from the desired shape. In the preferred embodiment, the segment 42 and paper 52 are heated for at least 15 minutes at a temperature between 260° F. and 300° F., but preferably between 265° F. and 280° F. due to the limitations, i.e., glass transition temperature of the carbon fiber epoxy composite. For the carbon fiber epoxy composite cuffs, the cuffs were preferably heated at 270° F. for 20 minutes in a convection oven. Aluminum or other metallic cuffs of similar shape could be sublimated at much higher temperatures, e.g., over 400° F., for shorter time periods due to the relatively high temperatures at which metals begin to soften or change shape. Likewise, any material that has a high transition temperature between the solid state and the softening state could be sublimated more quickly by use of a higher sublimation temperature. The segment 42 is preferably positioned within the oven so that it is placed in a non-stressed condition due to the weight of the clamps or is placed in a fixture to prevent the shape from being altered. In such a condition, little or no deformation of the segment 42 occurs even though the segment 42 may be heated above its glass transition temperature.

After the inks have been sublimated into the segment 42 and is cool enough to handle, the shrink tubing, the clamps 60, and the paper 52 are removed. The shrink tubing may be removed with cutters. Because of the re-positional spray adhesive used in securing the paper 52 to the segment 42, the paper 52 may be firmly secured to the segment 42 in some areas. One way in which the paper 52 may be removed along with the re-positional adhesive is by applying a lubricant or solvent over the sublimation paper 52. One suitable lubricant/solvent is WD-40 although other lubricants or solvents may be used. FIG. 4E depicts the segment 22 having sublimated graphics.

The invention permits a greater number of options to a user of an orthopedic device. Whereas before a user may be limited to only certain colors of a device, the invention allows a user to select from any of a number of graphic designs. Furthermore, in addition to a standard set of graphic designs, the invention also allows the user to customize his or her device with unique graphics. These graphics, for example, may be provided by the user and may be placed onto the sublimation paper 52 via sublimation printers. The graphics may be provided in any suitable format, such as with photographs, files, papers, or other formats from which the paper 52 may be prepared. The user can provide the desired design in any suitable manner, such as through file transfer or the Internet and may provide the design directly to the company providing the imaging or indirectly, such as through the user's physician.

The forgoing description of the preferred embodiments of the invention has been presented only for the purpose of illustration and description and is not intended to be exhaustive or to limit the invention to the precise forms disclosed. Many modifications and variations are possible in light of the above teaching.

For example, while the invention has been described with reference to the Defiance ACL knee-brace, the invention is not limited to just this orthopedic device. Instead, the invention may be applied to other orthopedic devices, which includes but is not limited to braces and supports, as well as to other three-dimensional objects. Moreover, even though the invention has been described with reference to an orthopedic device that permits movement at a joint, the invention is equally applicable to orthopedic devices that are intended to restrict movement of one or more joints and to orthopedic devices that are not located at a joint.

Furthermore, the invention may incorporate any suitable sublimation technique. Thus, the invention is not limited in the types of sublimation inks nor the type of paper or other substrate used as a carrier for the inks. For instance, the invention may be used with both thermal transfer sublimation and true sublimation. Additionally, the invention may be used with dye sublimation, wax thermal and wax thermal hybrid sublimation. Hanter Graphics of Poway, Calif. is a suitable printer for providing sublimation carrier paper with sublimation inks, although any other printer, any other source of carrier paper, and any other source sublimation ink may be used.

The invention is not limited in the type of graphics that may be applied to the object. In the example shown in FIGS. 4A to 4E, the sublimated graphics comprise a star pattern. The graphics applied to orthopedic devices may be appropriate for children and may include animals, popular characters, or other art work. With the invention, orthopedic devices may incorporate camouflage designs or may include logo information, such as the logo of a professional football team. As another example, the graphics that may be applied to orthopedic devices or other objects include graphics acquired from other sources, such as from a photograph, a file, or other document. As should be apparent, the invention may apply any type of graphics to an orthopedic device or other object.

While the graphics may be applied to both inner and outer surfaces of an object, such as the brace 20 shown in FIG. 2, the graphics need not be applied to all surfaces. For the brace 20, for instance, the graphics are preferably applied at least to an outer receiving surface since this surface remains visible when the device is worn by the user. Further, the graphics need not be applied on all visible surfaces but can be applied in only discrete portions of the object. Also, while a single sheet of sublimation paper is desirable since it allows for the application of an integral graphic design, multiple sheets of sublimation paper having the same graphics or different graphics may be applied to the same object.

The embodiments were chosen and described in order to explain the principles of the invention and their practical application so as to enable others skilled in the art to utilize the invention and various embodiments and with various modifications as are suited to the particular use contemplated.

We claim:

1. A method for sublimating ink onto a three-dimensional object, comprising:
providing the three-dimensional object having an outer receiving surface capable of receiving a sublimation ink, whereby the surface includes at least one complexity within its geometric profile;
placing sublimation carrier paper onto the outer receiving surface such that continuous intimate contact is formed therewith, whereby the carrier paper includes sublimation ink forming a graphic design thereon;
securing the carrier paper to the surface;
heating the carrier paper and the surface;
sublimating the ink to the surface thereby transferring the graphic design into the surface such that there is virtually no distortion about the complexity within the profile of the surface; and
removing the carrier paper from the surface.

2. The method as set forth in claim 1, wherein the three-dimensional object is an orthopedic device.

3. The method as set forth in claim 1, wherein the outer receiving surface is a integral with the three-dimensional object.

4. The method as set forth in claim 1, wherein the outer receiving surface is a coating.

5. The method as set forth in claim 1, further comprising curing the surface of the three-dimensional object prior to placing the carrier paper on the surface.

6. The method as set forth in claim 1, wherein placing comprises placing the carrier paper on more than one side of the three-dimensional object.

7. The method as set forth in claim 1, wherein the securing comprises applying an adhesive to the carrier paper prior to placing the carrier paper on the three-dimensional object.

8. The method as set forth in claim 1, wherein the securing comprises clamping areas of the carrier paper to the three-dimensional object.

9. The method as set forth in claim 1, wherein the securing comprises placing shrink wrap medium around a portion of the three-dimensional object.

10. The method as set forth in claim 1, wherein the three-dimensional object is formed of a polymer having a glass transition temperature and the heating comprises heating the three-dimensional object and the ink to a temperature less than the glass transition temperature.

11. The method as set forth in claim 1, wherein the heating comprises heating the three-dimensional object and the ink to a temperature above a glass transition temperature for the outer receiving surface.

12. The method as set forth in claim 1, wherein the three-dimensional object is part of an orthopedic device and the heating comprises heating the carrier paper and the surface to approximately 270° F.

13. An orthopedic device intended to be placed on a user, comprising:
a first portion intended to be secured to the user at a first position relative to a joint of the user;
a second portion intended to be secured to the user at a second position relative to the joint of the user;
a third portion connecting the first portion to the second portion and controlling motion of the first portion relative to the second portion;
wherein at least a part of one of the first portion, second portion, or third portion has sublimated graphics.

14. The orthopedic device as set forth in claim 13, wherein the first portion, second portion, and third portion are part of an orthopedic brace.

15. The orthopedic device as set forth in claim 13, wherein the at least one of the first portion, second portion, and third portion has a coating for receiving sublimated graphics.

16. The orthopedic device as set forth in claim 15, wherein the coating is comprised of polyurethane.

17. The orthopedic device as set forth in claim 15, wherein the at least one of the first portion, second portion, and third portion includes a colored layer underneath the coating.

18. The orthopedic device as set forth in claim 13, wherein the at least one of the first portion, second portion, and third portion has a curved surface for receiving the sublimated graphics.

19. The orthopedic device as set forth in claim 13, wherein the sublimated graphics are present on more than one side of the at least one of the first portion, second portion, and third portion.

20. A member for forming at least part of an orthopedic device, comprising:
a structure for being secured to a user;
the structure being shaped to fit the user and, when secured to the user, has a non-planar shape;
the structure being formed of a material suitable for receiving sublimated graphics;
wherein the structure includes sublimated ink in the material.

21. The member as set forth in claim 20, wherein the structure includes sublimated ink on multiples sides of the structure.

22. The orthopedic device as set forth in claim 13, wherein the sublimated graphics are included on a surface having at least one complexity and the graphics are substantially undistorted around the complexity.

23. An orthopedic device made from the method of claim 1.
